SEQUENCE LISTING (5 sequences)

SEQ ID NO: 1 - protein, 162 aa, homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 2 - protein, 213 aa, homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 456 aa, homo sapiens
QVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, homo sapiens
AIQLTQSPSSLSASVGDRVTITCRASQDIDSALAWYQQKPGKAPKILIHDASSLESGVPSRFSGSGSGTDFTLTISSLQPEDFATYYCQQFNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 447 aa, homo sapiens
EVQLVESGGGLVKPGGSLRLSCAASGFIFSSYSMNWVRQAPGKGLEWVSSITSGSYYIHYADSVKGRFTISRDNAKNSLYLQMNSLRAEDTAVYYCVRERGWGYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2016/0347833 A1
(43) Pub. Date: Dec. 1, 2016

(54) ANTIBODY PROCESS

(71) Applicant: NOVO NORDISK A/S, Bagsværd (DK)

(72) Inventors: Ole Elvang Jensen, Vanloese (DK); Mark Simon Chadfield, Gentofte (DK)

(21) Appl. No.: 15/114,655

(22) PCT Filed: Feb. 6, 2015
(86) PCT No.: PCT/EP2015/052536

§ 371 Date: Jul. 27, 2016

(30) Foreign Application Priority Data
Feb. 7, 2014 (EP) 14154266.2

(51) Int. Cl. C07K 16/24; C07K 1/18; C07K 1/34; C07K 1/36; C07K 1/20; C07K 1/22

(52) U.S. Cl. CPC C07K 16/244; C07K 1/20; C07K 1/22; C07K 1/34; C07K 1/36; C07K 1/18; C07K 2317/34

(57) ABSTRACT

The present invention provides a method for removing unwanted antibody aggregates, and/or antibody dimers, and/or antibody pre-monomers.

### TECHNICAL FIELD

The present invention concerns processes for producing high quality antibodies suitable for therapeutic use.

### BACKGROUND

Novel immuno-modulatory drugs based on biologics represent a revolution for many patients suffering from serious and chronic diseases such as e.g. various types of cancer and inflammatory diseases.

Many biologic drugs are based on antibodies. There is thus a need in the art for processes for providing high quality therapeutic antibodies of very high purity. It is known to use Protein A affinity chromatography, ion exchange chromatography, and hydrophobic interaction chromatography for purification of IgG (EP 746398). It is furthermore known to isolate monomeric IgG from misfolded antibody species using Protein A chromatography (U.S. Pat. No. 5,429,746). Purification of antibodies by HIC using a buffer with a very low pH (2.5-4.5) is known from U.S. Pat. No. 5,641,870 to be suitable for purification of F(ab′)2 antibodies.

In connection with purification of some antibodies, antibody aggregates such as, antibody dimers or high order aggregates may be formed—and in some cases they may be difficult to separate using analytical methods. There is thus a need in the art for improved methods for removal of antibody aggregates. In particular there is a need in the art for efficient methods for providing high quality (monomeric) antibodies from cell cultures, such as e.g. recombinant therapeutic antibodies.

### SUMMARY OF THE INVENTION

The present invention provides improved processes for purifying/isolating antibodies, in particular methods for removing unwanted antibody aggregates and/or antibody dimers and/or antibody pre-monomers from solutions containing monomeric antibodies, preferably without a significant reduction of (monomeric) antibody yield.

The present invention relates to a method for removing unwanted antibody aggregates, and/or antibody dimers, and/or antibody pre-monomers from an aqueous recombinant antibody solution, wherein said method comprises the step of purifying the (monomeric) antibodies using hydrophobic interaction chromatography (HIC). The invention furthermore relates to products resulting from such processes as well as use thereof.

### DESCRIPTION

The methods described herein are suitable for removal of antibody aggregates, and/or antibody dimers, and/or antibody pre-monomers, from aqueous (monomeric) antibody solutions. Preferably, the methods described herein result in recombinant therapeutic antibodies of a greater purity and/or more homogenous nature. In particular, the inventors have made the surprising discovery that the level of antibody aggregates, and/or antibody dimers, and/or antibody pre-monomers can be significantly reduced by employing a hydrophobic interaction chromatography purification step. The methods provided herein are in particular useful in connection with purification processes of recombinant antibodies from cell cultures.

### BRIEF DESCRIPTION OF DRAWINGS

FIG. 1: SEC-HPLC chromatogram showing the various anti IL-21 compounds (monomer and aggregates).

FIG. 2: The antibody pre-monomer impurity comprising two heavy chains (HC) and three light chains (LC), of which one is non-covalently attached (LC2HC2:LC). Based on the biophysical, spectroscopic and functional characteristics of LC2HC2:LC, the molecular structure shown is suggested, i.e. an antibody where an additional light chain has taken up the position normally occupied by a HC. The additional LC is bound exclusively via non-covalent interactions to another LC, which in turn is covalently bound to HC. The C-terminal cysteine in the non-covalently attached LC is capped by forming a disulfide bond with either glutathione or cysteine

FIG. 3: The FIGS. 3A and 3B shows the analysis of the eluate fraction from two HIC separations of antibody components as described in example 3. The scale to the left indicates antibody content, while the scale to the wright indication relative amounts of antibody aggregates. The monomer elutes first followed by the premonomer (hmwp3) and dimer (hmwp2) components of the antibody preparation. The higher order aggregates (hmwp1) elutes last.

### DEFINITIONS AND SEQUENCE INFORMATION

“Biologics”: A biologic drug is manufactured in a living system such as a microorganism, or plant or animal cells. Most biologics are large, complex molecules or mixtures of molecules. Many biologics are produced using recombinant DNA technology. It may be difficult, and sometimes impossible, to characterize a complex biologic drug by testing methods available in the laboratory, and some of the elements of a finished biologic drug may be unknown. Antibodies are an example of a biologic drug.

“Antibody”: The term “antibody”, “monoclonal antibody”, monomeric antibody, and “mAb” as used herein, is intended to refer to immunoglobulin molecules and fragments thereof that have the ability to specifically bind to an antigen. Monomer and monomeric antibody are herein used to describe antibodies of the regular format e.g. antibodies consisting of two light chains and two heavy chains. Antibodies herein are preferably recombinant antibodies. Full-length antibodies comprise four polypeptide chains, two heavy (H) chains and two light (L) chains interconnected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Antibodies can be in the form of different isotypes; e.g. IgG (e.g. IgG1, IgG2, IgG3, IgG4), IgGA1, IgA2, IgD, and IgE. A full-length antibody is normally bi-valent/di-valent, i.e. it has the capacity to bind to the antigen with both “arms”. In contrast, a monovalent antibody (e.g. a Fab fragment) comprises only one binding site specific for the antigen. The terms “human antibody”, as used herein, means antibodies having variable and constant regions derived from human germline immunoglobulin sequences. “Humanized antibodies” comprise CDR sequences from a non-human source (e.g. a mouse) grafted onto a human scaffold.

“Antibody aggregates”: Aggregates are complex of antibodies or parts of antibodies which differs in molecular weight from monomeric antibodies.

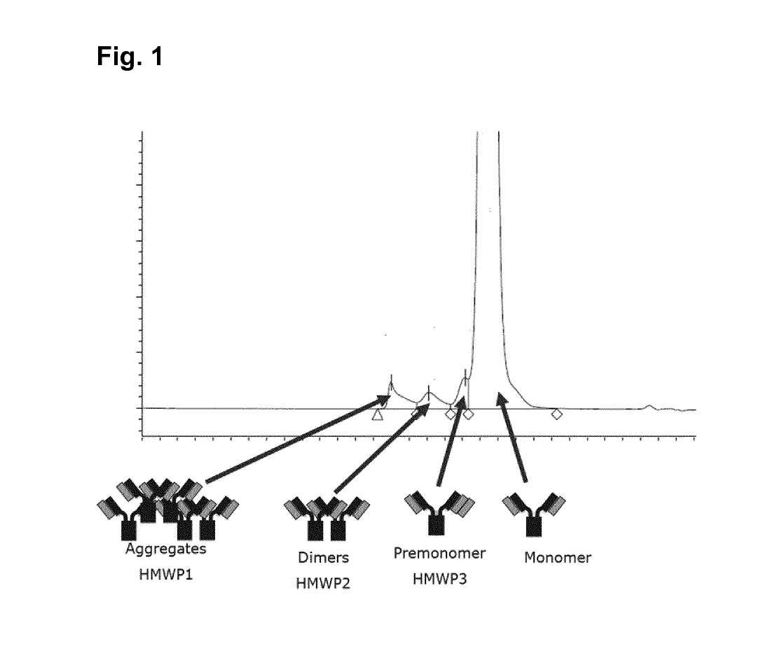

Examples of antibody aggregates include large aggregates including 3 or more antibody molecules which are non-covalently bonded. With reference to FIG. 1, such aggregates are referred to as HMWP1 or higher order aggregates. Antibody dimers are referred to by HMWP2 and describe complexes of two non-covalently bonded antibodies. The un-traditional aggregate described herein is referred to by HMWP3 or pre-monomer and is characterized as a complex of an antibody monomer and a non-covalently bonded light chain.

“Hydrophobic Interaction Chromatography (HIC)” refers to various techniques for the separation of complex mixtures that rely on the differential affinities of substances for a liquid mobile medium and for a stationary adsorbing medium through which they pass, such as paper, gelatin, or magnesia. Stationary column materials based on materials containing an aromatic hydrophobic group attached to the backbone, such as e.g. cross-linked agarose with a covalently coupled phenyl group are preferred. Agarose with about 2-15%, 3-12%, 4-10%, 4-8%, 5-7%, 2%, 3%, 4%, 5%, 6%, 7%, 8%, 9%, 10%, 11%, 12%, 13%, 14%, or 15% cross linking is preferred. Particularly preferred column materials are based on Phenyl Sepharose or materials having similar properties, such as e.g. “Phenyl Sepharose 6 Fast Flow® (low sub and high sub)” and “Phenyl Sepharose High Performance®” and Phenyl Sepharose CL-4B®, all trademarks of GE-HealthCare.Life Science.

“IL-21” is a type I cytokine, which exerts pleiotropic effects on both innate and adaptive immune responses. It is mainly produced by activated CD4+ T cells, follicular T cells and Natural killer cells. In addition, recent evidence suggests that Th17 cells can produce large amounts of IL-21. IL-21 increases the cytotoxicity of CD8+ T cells and can promote proliferation of CD8+ cells in the presence of antigens. IL-21 is induced by IL-6, a cytokine known to promote development of Th17 cells. IL-21 acts on T helper cells in an autocrine manner promoting its own production and supporting differentiation of T-helper cells into Th17 cells. IL-21 also acts on B-cells and increases antibody production. The ability of IL-21 to augment immunity has spurred substantial interest in the therapeutic use of IL-21. Animal studies have demonstrated a synergistic effect between IL-21 and tumour specific antibodies, which could suggest a future therapeutic use of IL-21 as a potentiator of anti-tumour antibodies. Furthermore, IL-21 plays a complex role in autoimmune diseases. The ability of IL-21 to promote Th17 development makes it a pro-inflammatory cytokine and IL-21 inhibitors are currently investigated for potential use in treatment of a range of different autoimmune diseases.

SEQ ID No 1: hIL-21 (incl. signal peptide spanning amino acids 1-29-mAb 5 epitope shown in bold; IL-21Rα binding site shown in underline; amino acid residues forming the mAb 14 epitope shown with lower case letters in italics)

MRSSPGNMERIVICLMVIFLGTLVHKSSSQGQDRHMIRMRQLIDIVDQLK
NYVNDLVPEFLPAPedvetnCewSAFSCFQKAQLKSANTGNNERIINVSI
KKLKRKPPSTNAGRRQkhrLTCPSCDSYEKKPPKEFLERFKS/
LQkmIhqhLSSRTHGSEDS

“IL-21 antibody”: Monoclonal (recombinant) antibodies specific for IL-21 are known in the art, for example from WO2007111714 and WO2010055366 (Zymo-Genetics, Inc.). In particular, WO2010055366 describes an anti-IL-21 antibody, designated by clone number 362.78.1.44 (“mAb 5”), which has a high affinity for its cognate antigen, and other desirable properties, showing specificity for human and cynomolgus monkey IL-21. Another antibody described therein is identified as clone number 366.328.10.63 (“mAb 14”). Preferred IL-21 antibodies herein are those that can compete/interfere with binding of the IL-21 receptor (IL-21R) to IL-21—examples of such antibodies are disclosed in WO12098113, including “mAb 5”. Other preferred IL-21 antibodies herein are those that can compete/interfere with binding of the common gamma chain to IL-21—examples of such antibodies are disclosed in WO201216402, incl. “mAb 14”. Antibodies disclosed in WO12098113 and WO2012164021 having the ability to compete/interfere with a receptor binding to IL-21 are thus incorporated herein.

Preferably, the IL-21 antibody herein binds to helix 1 and 3 of human IL-21. Preferably, the IL-21 antibody binds to a discontinuous epitope on IL-21, wherein said epitope comprises amino acids I37 to Y52 and N92 to P108 as set forth in SEQ ID NO 1. Preferably, the IL-21 antibody comprises the three CDR sequences as set forth in SEQ ID NO 2 and the three CDR sequences as set forth in SEQ ID NO 3. Preferably, the IL-21 antibody competes with γC (gamma chain) for binding to IL-21. Preferably, the IL-21 antibody binds to helix 2 and 4 of human IL-21. Preferably, the IL-21 antibody binds to an epitope comprising amino acids Glu 65, Asp 66, Val 67, Glu 68, Thr 69, Asn 70, Glu 72, Trp 73, Lys 117, His 118, Arg 119, Leu 143, Lys 146, Met 147, His 149, Gln 150, and His 151 as set forth in SEQ ID NO.1. Preferably, the IL-21 antibody comprises the three CDR sequences as set forth in SEQ ID NO 4 and the three CDR sequences as set forth in SEQ ID No 5.

SEQ ID No 2: “mAb 5”: light chain (signal peptide omitted—CDR sequences shown in bold/underline—constant region shown in lowercase letters—variable region in uppercase letters)

EIVLTQSPGTLSLSPGERATLSCRASQSVSSSYLAWYQQKPGQAPRLLIY
GASSRATGIPDRFSGSGSGTDFTLTISRLEPEDFAVYYCQQYGSWTFGQG
TKVEIKRtvaapsvfifppsdeqlksgtasvvcllnnfypreakvqwkvd
nalqsgnsqesvtecidskdstyslsstltlskadyekhkvyacevthqg
lsspvtksfnrgec

SEQ ID No 3: “mAb 5”: heavy chain of the IgG1 isotype (signal peptide omitted—CDR sequences shown in bold/underline—constant region shown in lowercase letters—variable region in uppercase letters)

QVQLVESGGGVVQPGRSLRLSCAASGFTFSSYGMHWVRQAPGKGLEWVAF
IWYDGSDKYYADSVKGRFTISRDNSKNTLYLQMNSLRAEDTAVYYCARDG
DSSDWYGDYYFGMDVWGQGTTVTVSSastkgpsvfplapsskstsggtaa
lgclykdyfpepvtvswnsgaltsgvhtfpavlgssglyslssvvtvpss
slgtgtyicnvnhkpsntkvdkkvepkscdkthtcppcpapeaegapsvf
lfppkpkdtlmisrtpevtcyvvdvshedpevkfnwpidgvevhnaktkp
reegynstyrvvsvltylhgdwlngkeykckvsnkalpssiektiskakg
gprepgvytlppsrdeltkngysltclvkgfypsdiavewesnggpenny
kttppvldsdgsfflyskltvdksrwgggnvfscsvmhealhnhytgksl
slspgk

SEQ ID No 4: “mAb14” light chain (signal peptide omitted—CDR sequences shown in bold/underline, constant region shown in lowercase letters)

AIQLTQSPSSLSASVGDRVTITCRASQDIDSALAWYQQKPGKAPKILIHD
ASSLESGVPSRFSGSGSGTDFTLTISSLQPEDFATYYCQQFNSYPYTFGQ
GTKLEIKRtvaapsvfifppsdeqlksgtasvvcllnnfypreakvqwkv
dnalqsgnsqesvteqdskdstyslsstltlskadyekhkvyacevthqg
lsspvtksfnrgec

SEQ ID No 5: “mAb14” heavy chain of the IgG4 isotype (signal peptide omitted—CDR sequences shown in bold/underline, constant region shown in lowercase letters)

EVQLVESGGGLVKPGGSLRLSCAASGFIFSSYSMNWVRQAPGKGLEWVSS
ITSGSYYIHYADSVKGRFTISRDNAKNSLYLQMNSLRAEDTAVYYCVRER
GWGYYGMDVWGQGTTVTVSSastkgpsvfplapcsrstsestaalgclvk
dyfpepvtvswnsgaltsgvhtfpavlcissglyslssvvtvpssslgtk
tytcnvdhkpsntkvdkrveskygppcpscpapeflggpsvflfppkpkd
tlmisrtpevtcvvvdvsqedpevqfnwyvdgvevhnaktkpreeqfnst
yrvvsvltvlhqdwlngkeykckvsnkglpssiektiskakgqprepqvy
tlppscieemtknqvsltclvkgfypsdiavewesngdpennykttppvl
dsdgsfflysrltvdksrwqegnvfscsvmhealhnhytqkslslslgk

### DETAILED DESCRIPTION

The present invention relates to a method for removing unwanted antibody aggregates, and/or antibody dimers, and/or antibody pre-monomers from an aqueous recombinant antibody solution, wherein said method comprises the step of purifying the monomeric antibodies using hydrophobic interaction chromatography (HIC). The invention furthermore relates to products resulting from such processes as well as use thereof. It should be clear that the term “remove” is used to described reduction of the amount or concentration of the antibody aggregates as a purification steps is rarely capable of removing the totality of the unwanted component(s).

Antibody products according to the inventions are such as compositions comprising at most 1% antibody pre-monomer aggregates, In particular compositions of an anti-IL-21 antibody comprising at most 1% antibody pre-monomer aggregates.

As described herein by the examples, preparation of an antibody composition requires multiple steps of which most are known to the person skilled in the art. The present invention describes the specific use of HIC to separate monomeric antibody molecules from antibody aggregates, which may have formed during the expression process or the initial purification of the antibody. Hydrophobic interaction chromatography has previously been used to separate monomeric antibodies from dimers and higher order aggregates. As described herein the inventors of the present application have identified a further species of antibody aggregates that as described above has been named pre-monomer which include an antibody monomer and a light chain non-covalently bonded hereto. The attachment of a light chain only changes the Molecular weight slightly compared to the monomeric antibody and therefore represent a greater challenge in terms of purification of the monomeric antibody for therapeutic use. As seen in FIG. 1, the pre-monomer elutes right before (and together with) the main peak of monomeric antibody when analysed by SEC-HPLC, and is thus clearly distinguished from the dimers and higher aggregates of antibodies.

An antibody preparation is a composition comprising the monomeric antibody of interest as well as antibody aggregates, such as the pre-monomer (as described herein), antibody dimers and higher order antibody aggregates as components of the preparation.

In one embodiment the antibody of interest, is a full-length antibody e.g. the preparation should include a majority of this particular full-length antibody whereas the antibody aggregates are considered impurities.

The antibody preparation may as described be obtained from a recombinant source and processed by one more methods aimed to at least partially purify the antibody of interest. Such methods are known in the art and includes such as protein A purification, filtering and other chromatographic methods.

The method according to the invention comprises the steps of;
i) obtaining an antibody preparation comprising a pre-monomer component
ii) loading said antibody preparation to a hydrophobic interaction chromatography column allowing binding of the antibody and the pre-monomer to the column material
iii) sequentially eluting the antibody components of the preparation and
iv) selecting eluate fractions that do not comprise substantial amount of the pre-monomer.

Suitable types of hydrophobic interaction chromatography columns are described herein above. It is preferred that the binding capacity the HIC column material is at least such as around 50 g antibody/L or at least 60 g antibody/L. In preferred embodiments the column material comprises 4-8% cross-linked agarose comprising a covalently coupled phenyl group. In further embodiments the HIC column material comprises about 15-35, 15-30, 20-35, 20-30, 15, 16, 17, 18, 19, 20, 21, 22, 23, 24, 25, 26, 27, 28, 29, 30, 31, 32, 33, 34, or 35 μmol phenyl pr. ml gel, or 20/40 μmol/ml. It is estimated that 20-25 μmol phenyl pr. ml gel corresponds to the estimated capacity for Sepharose High Performa while 40 μmol phenyl pr. ml gel corresponds to the estimated capacity for Sepharose 6 FastFlow®).

The length of the HIC column may influences the quality of the HIC purification step, wherefore a longer column may be preferred, such as a HIC column of at least 5 cm, such as at least 8 cm, such as at least 12 cm. In a further embodiment the HIC column is 5-50 cm, such as 8-25 cm, such as 10-20 cm, such as 8-15 cm.

In one embodiment the loading (step b) is performed in the presence of ammonium sulphate ((NH₄)₂SO₄), such as with a concentration of ammonium sulphate about 0.8-1.5 mol/kg, preferably 1.2-1.0 or 1.1-0.9 mol/kg,

The elution may in such embodiments be performed by decreasing the ammonium sulphate concentration, such as by using a linear gradient of ammonium sulphate to 0 mol/kg.

The use of HIC has demonstrated the ability to separate the antibody components of the preparation as the various components elute in a sequential manner. Although elution is described as sequential, there may be some overlap where the eluted fractions comprise more than one antibody component, but the majority of the individual components should elute in different fractions.

In one embodiment the monomeric antibody components elutes as the first antibody component. In such examples the antibody solution of interest may be obtained by collecting the fractions eluting prior to the fractions including the majority of pre-monomers and antibody dimers.

In such embodiments the pre-monomer may be excluded from the antibody composition by deselecting subsequent eluate fractions. In one embodiment the pre-monomer (and other aggregates) are removed from the antibody solution by selecting only the fractions eluting prior to the majority of the pre-monomer.

According to the invention eluate fraction not comprising substantial amount of the pre-monomer are selected. Substantial amount is an amount that would be consider not acceptable for the purpose of purifying the monomeric antibody.

In further embodiments elute fractions comprising a substantial amount any aggregate are deselected. Likewise if the sum of aggregates is substantial, such elute fractions are to be deselected. In most cases substantial amount of aggregates (including pre-monomers) would be present if aggregates represent 5% of the protein content.

As described in the examples a threshold content of one or more antibody aggregates may determine which eluate fractions are useful to obtain the antibody composition of interest. In a preferred embodiment the total amount of antibody aggregates is 1% or less.

A method according to the invention, wherein said method comprises the following steps:
a) expressing an antibody in a host cell, wherein said host cell comprises a vector (alternatively two vectors encoding the heavy chain and the lights chain, respectively) encoding said antibody,
b) collecting cell media, comprising said antibody, from step (a),
c) binding antibodies from the cell media obtained in step (b) on a protein A affinity column, and eluting said antibodies with about 10 mmol/kg formic acid (such as e.g. 5-15 7-13, 8-12, 5, 6, 7, 8, 9, 10, 11, 12, 13, 14, or 15 mmol formic acid/kg) at a pH of about 3.5 (such as e.g. 2.5-4.5, 3-4, 2.5, 2.6, 2.7, 2.8, 2.9, 3.0, 3.1, 3.2, 3.3, 3.4, 3.5, 3.6, 3.7, 3.8, 3.9, or 4.0),
d) optionally adjusting pH of the eluate obtained in step (c) to 3.7 in order to inactivate any viruses, (viruses may also be inactivated using other methods),
e) loading the eluate obtained in step (c) or (d) on a cation exchange chromatography column (CIEX), after pH adjustment as needed, and eluting said antibodies with a salt gradient (e.g. a NaCl gradient) optionally using e.g. POPOS 50 HS, and optionally the eluate may be filtered using pore sizes of 0.45 μm, optionally in combination with pH adjustment,
f) subjecting the eluate obtained in step (e) to virus filtration, using e.g. a Planova® 20N virus filter,
g) pumping the filtered eluate obtained in step (f) through a flow though membrane (such as a Q membrane), and optionally adjusting pH and conductivity prior to filtering on said flow through membrane,
h) loading the flow through product obtained in step (g) on a hydrophobic interaction chromatography column in the presence of an ammonium sulphate concentration of about 0.8-1.5 mol/kg, preferably 1.0 mol/kg,
i) eluting said antibodies with a decreasing ammonium sulphate gradient,
j) concentrating said antibodies in the eluate obtained in step (i) by ultrafiltration
k) followed by diafiltration and further ultrafiltration, optionally in the presence of sucrose.

As described herein, the method may be used for preparing an antibody composition comprising at most 1% antibody aggregates (HMWP total), wherein the method comprises removing antibody pre-monomers from an aqueous recombinant antibody solution by using hydrophobic interaction chromatography (HIC). In a further embodiment both pre-monomers and antibody dimers are separated from (correctly folded) monomeric antibodies using hydrophobic interaction chromatography (HIC). As described herein above, the method may be further described by specification of the individual steps as described herein above.

An aspect of the invention relates to a pharmaceutical product or formulation obtained by (or obtainable by) a process according to the invention. In one embodiment, the invention relates to a pharmaceutical product, wherein the amount of antibody aggregates is 1% or less of the total amount of antibody. In most instances the amount of antibody aggregates such as antibody dimers and antibody pre-monomers can be measured by SEC-HPLC. An residual level of aggregates below the level of detection is rarely expected, but of course an attractive embodiment. It is further preferred that the pharmaceutical product according to the invention, include no detectable virus. Virus counts can be measured using methods well known in the art.

The resulting antibody composition may be referred to as a homogenous antibody composition which may be used either directly as a pharmaceutical product or used for the preparation of a pharmaceutical product. A final antibody product may be referred to as a pharmaceutical formulation.

According to the invention the antibody composition, antibody product, pharmaceutical composition or pharmaceutical product obtained by the method described herein, may be used for in a method of treatment. The compositions and products may accordingly be for use in a method of treatment of an inflammatory disease.

The antibody compositions and products disclosed herein are for use in a method of treatment of inflammatory diseases and conditions, in particular anti-inflammatory diseases, such as e.g. psoriasis, type I diabetes, Grave's disease, Inflammatory bowel disease (IBD), Crohn's disease, ulcerative colitis, irritable bowel syndrome, multiple sclerosis, rheumatoid arthritis (RA), autoimmune myocarditis, Kawasaki disease, coronary artery disease, chronic obstructive pulmonary disease, interstitial lung disease, autoimmune thyroiditis, systemic lupus erythematosus (SLE), scleroderma, systemic sclerosis, psoriatic arthritis, osteoarthritis, atoptic dermatitis, vitiligo, graft vs. host disease, Sjoogrens's syndrome, autoimmune nephritis, Goodpasture's syndrome, chronic inflammatory demyeliniating polyneutopathy, allergy, asthma and other autoimmune diseases. The antibody products herein are furthermore suitable for treatment of various cancer types, e.g. carcinomas, sarcomas, lymphomas, leukemia, germ cell tumors, and blastomas.

The invention as described herein is exemplified, but not limited, in the following list of embodiments:

### EMBODIMENTS

1. A method for removing antibody dimers and/or antibody pre-monomers from an aqueous recombinant antibody solution, wherein said method comprises the step of separating the dimers and/or pre-monomers from (correctly folded) antibodies using hydrophobic interaction chromatography (HIC).
2. A method for removing antibody pre-monomers from an aqueous recombinant antibody solution, wherein said method comprises the step of separating pre-monomers from monomeric (correctly folded) antibodies using hydrophobic interaction chromatography (HIC).
3. A method according to the invention, wherein said antibody is an IL-21 antibody.
4. A method according to the invention, wherein the antibody comprises the three CDR sequences as set forth in SEQ ID NO 2 and the three CDR sequences as set forth in SEQ ID NO 3. Alternatively said antibody comprises the VH/VL sequences set forth in SEQ ID NOs 2+3.
5. A method according to the invention, wherein the antibody is a full-length antibody.
6. A method according to the invention, wherein the HIC column material has a binding capacity of 50 g antibody/L (or more), preferably 60 g antibody/L (or more).
7. A method according to the invention, wherein the HIC column material comprises 4-8% cross-linked agarose comprising a covalently coupled phenyl group, preferably 6%.
8. A method according to the invention, wherein the HIC column material comprises about 15-35, 15-30, 20-35, 20-30, 15, 16, 17, 18, 19, 20, 21, 22, 23, 24, 25, 26, 27, 28, 29, 30, 31, 32, 33, 34, or 35 μmol phenyl pr. ml gel, or 20/40 μmol/ml (20-25 μmol phenyl pr. ml gel corresponds to the estimated capacity for Sepharose High Performance®; 40 μmol phenyl pr. ml gel corresponds to the estimated capacity for Sepharose 6 FastFlow®).
9. A method according to the invention, wherein the length of the HIC column is at least 5 cm, such as at least 8 cm, such as at least 12 cm.
10. A method according to the invention, wherein the length of the HIC column is 5-50 cm, such as 8-25 cm, such as 10-20 cm, such as 8-15 cm.
11. A method according to the invention, wherein said method comprises the step of loading said antibody on the HIC column in the presence of a high ammonium sulphate concentration (about 1 mol/kg, or at least about 0.8 mol/kg, or at least about 0.8 mol/kg and not more than 1.5 mol/kg) and subsequently eluting said antibody from the HIC column with a decreasing ammonium sulphate gradient.
12. The method according to any of the previous claims, wherein the monomeric antibody is obtained in the eluate fractions obtained when the ammonium sulphate concentration is decreasing from around 0.8 mol/kg to 0.4 mol/kg.
13. A method according to the invention, wherein the pre-monomer is removed by deselecting the eluate fractions when the accumulation of pre-monomers reaches around 1%.
14. The method according to the invention where the monomeric antibody is obtained in the eluate fractions obtained when the OD280 is between 0.2 on the front edge and 8.0 on the tailing edge.
15. The method according to the invention where the antibody composition of interest is obtained in the fractions eluting prior to the fractions including a high content of pre-monomer and antibody dimers.
16. The method according to the invention where the antibody composition of interest is obtained in the fractions eluting prior to the fractions including a high content of pre-monomer.
17. A method according to the invention, wherein said method results in reduction of virus particles.
18. A method according to the invention, wherein said HIC purification is performed as the last step prior to concentration of said antibody, and/or prior to diafiltration and/or prior to freeze drying and/or prior to formulation of said antibody.
19. A method according to the invention comprising the steps of;
   i) obtaining an antibody preparation comprising a pre-monomer component
   ii) loading said antibody preparation to a hydrophobic interaction chromatography column
   iii) allowing binding of the antibody and the pre-monomer to the column material
   iv) sequentially eluting the antibody components of the preparation and
   v) selecting eluate fractions that do not comprise substantial amount of the pre-monomer.
20. A method according to the invention, wherein said method comprises the following steps:
   a. expressing an antibody in a host cell, wherein said host cell comprises a vector (alternatively two vectors encoding the heavy chain and the lights chain, respectively) encoding said antibody,
   b. collecting cell media, comprising said antibody, from step (a),
   c. binding antibodies from the cell media obtained in step (b) on a protein A affinity column, and eluting said antibodies with about 10 mmol/kg formic acid (such as e.g. 5-15 7-13, 8-12, 5, 6, 7, 8, 9, 10, 11, 12, 13, 14, or 15 mmol formic acid/kg) at a pH of about 3.5 (such as e.g. 2.5-4.5, 3-4, 2.5, 2.6, 2.7, 2.8, 2.9, 3.0, 3.1, 3.2, 3.3, 3.4, 3.5, 3.6, 3.7, 3.8, 3.9, or 4.0),
   d. optionally adjusting pH of the eluate obtained in step (c) to 3.7 in order to inactive any viruses, (viruses may also be inactived using other methods),
   e. loading the eluate obtained in step (c) or (d) on a cation exchange chromatography column (CIEX), and eluting said antibodies with a salt gradient (e.g. a NaCl gradient) optionally using e.g. POPOS 50 HS, and optionally the eluate may be filtered using pore sizes of 0.45 μm, optionally in combination with pH adjustment,
   f. subjecting the eluate obtained in step (e) to virus filtration, using e.g. a Planova® 20N virus filter,
   g. pumping the filtered eluate obtained in step (f) trough a flow though membrane (e.g. a Q membrane), and optionally adjusting pH and conductivity prior to filtering on said flow through membrane,
   h. loading the flow through product obtained in step (g) on a hydrophobic interaction chromatography column in the presence of an ammonium sulphate concentration of about 0.8-1.5 mol/kg, preferably 1.0 mol/kg,
   i. eluting said antibodies with a decreasing ammonium sulphate gradient,
   j. concentrating said antibodies in the eluate obtained in step (i) by ultrafiltration
   k. optionally followed by diafiltration and further ultrafiltration, possibly in the presence of sucrose, and
   l. adding surfactant to the concentrated eluate obtained in step (j) or the eluate of step (k) and optionally filtering said eluate, and optionally freeze drying said filtered eluate.
21. A method for preparing an antibody composition comprising at most 1% antibody aggregates (HMWP total), comprising removing antibody pre-monomers from an aqueous recombinant antibody solution by using hydrophobic interaction chromatography (HIC).
22. The method according to embodiment 21, wherein pre-monomers and antibody dimers are separated from (correctly folded) antibodies using hydrophobic interaction chromatography (HIC)
23. The method according to embodiment 21 or 22, comprising one or more of the features of embodiments 3-20.
24. A pharmaceutical product obtained by (or obtainable by) a process according to the invention.
25. A pharmaceutical product according to the invention, wherein the amount of antibody aggregates is 1% or less of the total amount of antibody.
26. A pharmaceutical product according to the invention, wherein no detectable virus is present. Virus counts can be measured using methods well known in the art.
27. A pharmaceutical formulation comprising a pharmaceutical product according to the invention.
28. Use of pharmaceutical formulation according to the invention for treating an inflammatory disease.
29. Use of a pharmaceutical formulation according to the invention for treating an inflammatory disease selected from type 1 diabetes, Inflammatory Bowel disease, Crohns disease, psoreasis, graft vs. host disease, and SLE.
30. Use of a pharmaceutical formulation according to the invention for treating cancer.
31. A pharmaceutical product according to the invention for use in a method of treatment an inflammatory disease.
32. A pharmaceutical product according to the invention for use in a method of treatment of an inflammatory disease selected from type 1 diabetes, Inflammatory Bowel disease, Crohns disease, psoreasis, graft vs. host disease, and SLE.
33. A pharmaceutical product according to the invention for use in a method for treatment of cancer.

### EXAMPLES

### Example 1

A purification process has proven useful for purifying various recombinant antibodies from cell culture on an industrial scale. This process can be schematisized as follows:

This relatively simple purification process resulted in high antibody yields of a very high purity and was therefore very efficient for purifying different recombinant therapeutic antibodies on an industrial scale see also WO2009/138484 with further information on specific examples.

In connection with purification of IL-21 antibodies however—in particular antibodies having the CDR sequences of clone 362.78.1.44 disclosed in WO2010055366 (mAb 5″)—the resulting purified IL-21 antibodies surprisingly contained unwanted high molecular weight proteins (HMWP) in amounts of around 2-3%. It furthermore turned out that the HMWP content accumulated/increased over time—in particular the dimer concentration. Some of these aggregates were apparently difficult to separate without significantly reducing the antibody yield. Various purification methods were employed in the attempt to remove HMWP (incl. antibody dimers and antibody pre-monomers) to amounts around or below 1% without significantly reducing antibody yield (various cation exchange methods such as Poros® HS 50, parameters investigated like flow, load, gradient length, cutting criteria, Fractogel® EMD COO, Fractogel® EMD SO₃, Capto™ Adhere, Capto™ MMC, and certain HIC resins, TSK-Gel® Phenyl-5PW). These methods all resulted in a significant (and unacceptable) reduction in antibody yield. SEC HPLC analysis of the aggregates revealed that they comprised antibody dimers and antibody pre-monomers.

It was thus desirable to provide anti IL-21 compositions of improved purity, said antibodies having reduced amounts of unwanted aggregates such as dimers and/or pre-monomers. A solution to this problem was discovered by the inventors as described herein. HIC is known to be useful in some cases for reducing antibody dimers and high order aggregates in antibody purificaiton processes. Various resins were therfore investigated in combination with binding in high ammonium sulphate concentration and eluting in a gradient to zero ammonium sulphate.

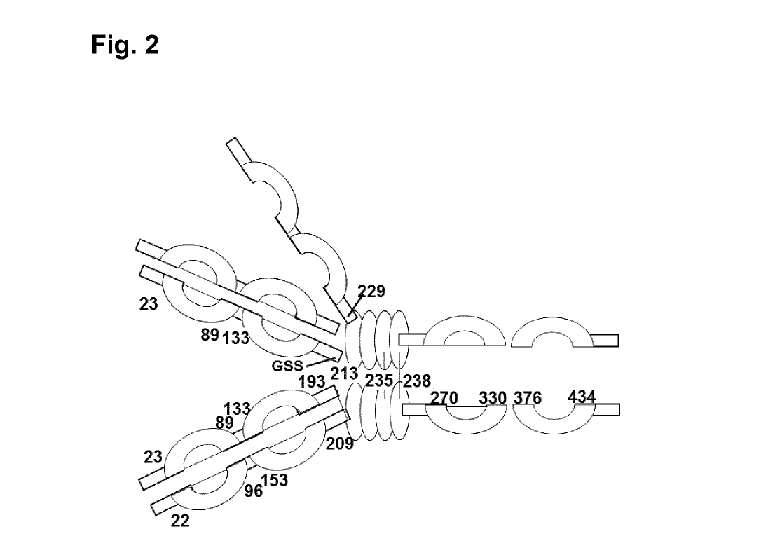

Some of the impurities in e.g. the Anti-IL-21 process are the HMWP (high molecular weight proteins) collectively referred to as aggregates. They comprise HMWP1=higher order aggregates, HMWP2=antibody dimers, HMWP3=antibody pre-monomer (FIG. 2). The HMWP1 is removed during the CIEX step, but the general process did not result in sufficient reduction of the pre-monomer and dimers. There appears to be no selectivity towards the pre-monomer (HMWP3) at all with CIEX (POROS 50 HS, or Fractogel COO) (See also Example 4).

### Example 2

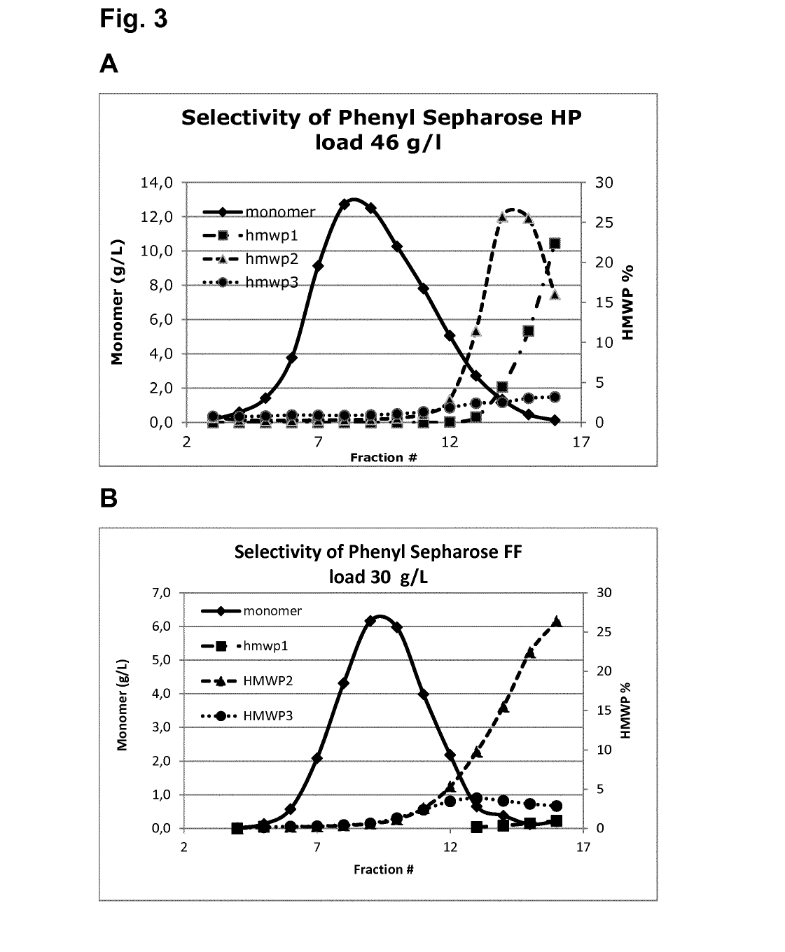

Employment of a HIC purificaiton step using a column material comprising Phenyl Sepharose™ FF and/or Phenyl Sepharose™ HP according to the manufactures instructions surprisingly demonstrated the desired selectivity towards the pre-monomer and the dimer—the pre-monomer elutes at the back of the monomer peak together with the other aggregates (see FIG. 3 and description below). A gradient with decreasing ammonium sulphate concentrations is preferably used for the HIC elution step. The Phenyl Sepharose™ HP material has the advantage of a high binding capacity (more than 60 g mAb/L packing material). This type of column material has a relatively low density of phenyl groups (25 μmol/ml) and it is thus surprising that this material has superior properties in relation to Ab binding capacity in comparison with e.g. Phenyl Sepharose™ FF high sub having a phenyle density of 40 μmol/ml.

The HIC step preferably takes place prior to the final UF/DF step (prior to formulation of the active pharmaceutical ingredient—in this case a recombinant therapeutic antibody). The HIC step preferably takes place as one of the last steps before freeze drying and/or preparation of the pharmaceutical formulation of the antibody in order to avoid accumulation of dimer during the purification process. A buffer change, such as a step of diafiltration may be used after HIC to ensure that the antibody is in a solution suitable for freeze drying.

Dimer formation of the IL-21 antibody apparently occurs spontaneously during the production process. Therefore, the HIC step/HIC purification process can be used for purifying antibodies having a tendency to form aggregates.

In addition to reducing the dimer formation associated with the production (and purification) of this protein, the HIC step also potentially results in viral clearance/reduction thus also potentially improving safety of the purified antibody products.

### HIC Purification Step in Purification of Anti-IL-21:

### Solvents:

| Solvent | S1 | S1b | S2 | S3 |
|---|---|---|---|---|
| NaOH | | | | 1.0 M |
| Na₂HPO₄ x2H₂O | 25 mM | 25 mM | 25 mM | |
| (NH₄)₂SO₄ | 1.0 M | 1.2 M | 0 | |
| (AmSO₄) | | | | |
| pH | 7.0 | 7.0 | 7 | |

### Method

A column (1 cm diameter, 13 cm length) was packed with 10 ml Phenyl Sepharose HP was equilibrated with 5 column volume (CV) S1, flow rate was 15 CV/h.

The loading solution was prepared by adding AmSO₄ to the antibody solution obtained from the previous cation exchange step. To 110 ml of the starting antibody preparation (6.6 g/L) is added 16.72 g AmSO₄ yielding an antibody concentration of 5.57 g/L. 105 ml of the loading solution is loaded with a flow rate 9 CV/h. The resulting load is 58 g of antibody per L resin.

Following load, the column is washed with 5CV S1 (flow rate 15 CV/h) and elution is effected by a gradient from 100% S1 to 100% S2 over 15 CV (flow rate 15 CV/h) followed by 5 CV of S2 (flow rate 15 CV/h). Fraction was collected depending OD, starting at OD 0.2 at the front and at 8.0 at the tailing edge. Finally the column was sanitised with 2 CV S3 flow rate 3 CV/h. The protein content is measured using OD280, defined as UV absorption at 280 nm over 1 cm. To minimize the amounts of the pre-monomer (and other aggregates) the cut-off was to be below 1% HMWP and the antibody product of interest may thus be obtained by collection the fractions eluting from the column with an OD280 of 0.2 on the front edge together with the fractions eluting with an OD280 of up to 8.0 on the tailing edge.

The percentage of monomers and aggregates of the resulting elute was analysed. The analytical procedure is a size-exclusion chromatography (SE-HPLC) test, where the samples are analysed using a TSK G3000 SWxl column, isocratic elution using a sodium phosphate/isopropanol buffer and subsequent UV detection at 280 nm. % HMWP is calculated relative to the total integrated area. The result is included in the table here below, demonstrating that the HIC step removes the majority of the aggregates reducing the total concentration of aggregates to 0.96%.

| | | Protein conc. | HMWPtot | HMWP1 | HMWP2 | HMWP3 |
|---|---|---|---|---|---|---|
| | OD280 | (mg/m)l | (weight %?) | (weight %?) | (weight %?) | (weight %?) |
| Antibody loading | 8.811 | 5.5 | 6.3 | 2.73 | 2.27 | 1.3 |
| solution | | | | | | |
| Eluate | 8.968 | 5.6 | 0.96 | 0.02 | 0.18 | 0.75 |

### Example 3

To further analyse the effect of HIC and to further analyse which fractions of eluate to collect to minimize content of the pre-monomer while maximizing yield of antibody a purification using Phenyl Sepharose HP and a protein load of 46 g/L was performed as described above.

The loading solution was prepared using 36.8 ml protein A virus inactived antibody preparation (concentration of 6.6 g/L) by addition of 5.6 g AmSO₄.

A column of 3.4 cm in length and a diameter of 1 cm was packed with 2.7 ml Phenyl Sepharose HP and equilibrated with 5 CV S1b and loaded with 26.33 ml of the loading solution (4.77 g antibody/L) corresponding to a total load of 46 g antibody (anti-IL-21) per L resin.

The column is washed with 5 CV S1b and eluted with a linear gradient from 100% S1b to 100% S2 over 10 CV followed by 6 CV S2. 2 ml fractions were collected during elution. The antibody preparation was loaded in buffer including 1 mol/kg (NH₄)₂SO₄. Elution of protein is obtained in fractions 3-15 by a linear decrease of the (NH₄)₂SO₄ concentration reaching 0 mol/kg at fraction 15. Table 2 shows the protein concentration in each fraction and the percentage of HMWP1, HMWP2 and HMWP3. The purification process is illustrated in FIG. 3A and while HMWP1 and HMWP2 shows a clear peak well separated from the monomeric antibody, the pre-monomer (HMWP3) elutes in various fractions.

TABLE 2
Protein analysis of HIC fractions from Phenyl Sepharose HP
purification

| | Protein | HMWP % | | |
|---|---|---|---|---|
| Fraction # | Conc. g/l | HMWP1 % | HMWP2 % | HMWP3 % |
| 3 | 0.17 | 0 | 0.66 | 0.76 |
| 4 | 0.60 | 0 | 0.29 | 0.73 |
| 5 | 1.42 | 0 | 0.26 | 0.79 |
| 6 | 3.77 | 0 | 0.26 | 0.92 |
| 7 | 9.13 | 0 | 0.29 | 0.89 |
| 8 | 12.72 | 0 | 0.32 | 0.87 |
| 9 | 12.50 | 0 | 0.38 | 0.93 |
| 10 | 10.26 | 0 | 0.55 | 1.07 |
| 11 | 7.81 | 0 | 1.04 | 1.32 |
| 12 | 5.06 | 0.07 | 2.84 | 1.84 |
| 13 | 2.72 | 0.67 | 11.45 | 2.4 |
| 14 | 1.33 | 4.45 | 25.73 | 2.51 |
| 15 | 0.46 | 11.44 | 25.52 | 3.02 |
| 16 | 0.13 | 22.37 | 16.02 | 3.18 |

In similar way purification using Phenyl Sepharose 6 FF and a protein load of 37 g/L was performed and analysed.

A column of 11 cm in length and a diameter of 0.5 cm was packed with 2.1 ml Phenyl Sepharose 6 FF and equilibrated with 5 CV S1 b.

A loading solution was prepared from a protein A purified and virus inactivated antibody preparation (6.6 g/L) as above 14.2 ml was mixed with 5.8 ml of 3 M AmSO4, pH 7. 13.7 ml of the loading solution (4.77 g/L) was loaded on the column corresponding to a load of 30 g antibody per L resin.

After loading the column is washed with 5 CV S1b and eluted with a linear gradient from 100% S1b to 100% S2 over 10 CV followed by 10 CV S2. During the elution 2 ml fractions were collected. A gradient between fraction 2 to 12 from 1.2 mol/kg to 0 mol/kg (NH₄)₂SO₄ in 25 mmol/kg Na₂HPO₄, pH 7 was applied. The protein analysis of the obtained eluted fractions is shown in table 3.

TABLE 3
Protein analysis of HIC fractions from Phenyl Sepharose 6 FF
purification

| | Protein | HMWP % | | |
|---|---|---|---|---|
| Fraction # | Conc. g/L | HMWP1 % | HMWP2 % | HMWP3 % |
| 4 | 0.01 | 0 | 0 | 0 |
| 5 | 0.13 | 0 | 0 | 0.15 |
| 6 | 0.57 | 0 | 0.22 | 0.26 |
| 7 | 2.09 | 0 | 0.25 | 0.29 |
| 8 | 4.32 | 0 | 0.38 | 0.45 |
| 9 | 6.16 | 0 | 0.6 | 0.65 |
| 10 | 5.98 | 0 | 1.14 | 1.32 |
| 11 | 3.99 | 0 | 2.57 | 2.39 |
| 12 | 2.18 | 0 | 5.32 | 3.46 |
| 13 | 0.66 | 0.19 | 9.77 | 3.86 |
| 14 | 0.38 | 0.36 | 15.49 | 3.52 |
| 15 | 0.12 | 0.66 | 22.41 | 3.13 |
| 16 | 0.21 | 0.98 | 26.37 | 2.86 |

To minimize the amounts of the pre-monomer the cut-off was to be below such as 1% accumulated HMWP2 and 3 correlating with an (NH₄)₂SO₄ concentration of around 0.4 mol/kg. The product of interest may thus, as described before, be collected by obtaining the fractions eluting from the column with an with an OD280 of 0.2 on the front edge together with the fractions eluting with an OD280 of up to 8.0 on the tailing edge, thereby discharging fractions with a high content of pre-monomer and dimers.

### Example 4

A large scale experiment was performed. Starting from an antibody preparation obtained after anion exchange chromatography.

To a total of 1411 g of antibody in a composition of a total weight of 391 kg was added 59.58 kg solid (NH4)₂SO₄, the pH was adjusted to 7.0 with 1.0 mol/kg NaOH.

A column packed with 10.73 l Phenyl Sepharose HP (length 15.6 cm, diameter 29.6 cm) and equilibrated with 3.8 CV S1, flow 80 l/h, was loaded with 201.8 l of the loading solution described above with a flow of 60 l/h, corresponding to 62.9 g of anti IL_21 antibody per L resin. The column was washed with 5 CV S1, 80 l/h, and eluted with a linear gradient from 100% S1 to 100% S2 over 15 CV, 80 L/h, followed by 5 CV S2, flow 80 L/h. The column was sanitised with 2 CV S3, flow 20 l/h, and finally rinsed with 2 CV water, flow 80 l/h. The antibody product was collected from OD280 at 0.2 in the front edge to 8.0 on the tailing edge. The resulting purity was 0.8% HMWP compared to a purity of the loading solution of 2.9% HMWP.

Based on the above it is also noted that the length of the column influence the results obtained in such a way that a longer column (as used in this experiment) provide a better resolution than the shorter used in Example 3.

### Example 5

Initially an experiment was performed to evaluate the usability of CIEX POROS 50 HS for obtaining an antibody composition free of the pre-monomer.

Buffer A: 25 mmol/kg NaAcetate, pH 5 and Buffer B: 25 mmol/kg NaAcetate+300 mmol/kg NaCl, pH 5.

The purification was performed with a 5 ml column (1 cmD×20 cmL) loaded with 20 g alL21/l resin. The column was equilibrated with 5 cv Buffer A, loaded and eluted with a gradient from 100% Buffer A to 100% Buffer B over 20 CV followed by 5 CV buffer B. Elution of the monomeric antibody was obtained using a gradient between fraction 25 and 37 going from 50%-72.5% of buffer B. The separation was analyzed as above and as can be seen in FIG. 4 the pre-monomer (HMWP3) elutes in front of monomer peak, while dimer elutes in tail of monomer peak. Due to the early elution of HMWP3 it was only possible to obtain very few fractions with a low content of aggregates (pre-monomers, dimers and/or high order aggregates) resulting in a very poor yield.

TABLE 4

| | Protein | HMWP % | | |
|---|---|---|---|---|
| Fraction # | Conc. g/l | HMWP1 | HMWP2 | HMWP3 |
| 25 | 0.07 | | | |
| 26 | 0.20 | 0 | 0.26 | 0.44 |
| 27 | 0.56 | 0 | 0.3 | 0.37 |
| 28 | 1.20 | 0 | 0.29 | 0.41 |
| 29 | 2.90 | 0 | 0.34 | 0.49 |
| 30 | 5.83 | 0 | 0.38 | 0.36 |
| 31 | 8.03 | 0 | 0.45 | 0.23 |
| 32 | 9.22 | 0 | 0.49 | 0.14 |
| 33 | 10.30 | 0 | 0.91 | 0.1 |
| 34 | 7.49 | 0 | 2.78 | 0 |
| 35 | 2.29 | 0 | 11.51 | 0 |
| 36 | 0.74 | 0 | 28.31 | 0 |
| 37 | 0.31 | | | |

### Example 6

### Over all Description of an Antibody Purification Process Including the HIC Step

Protein A derivate step is developed in order to concentrate the clarified culture broth and function as a very specific affinity capture step for mAb. Furthermore, the step includes virus inactivation (enveloped vira) by adjusting pH to 3.7.

CIEX is a cation exchange step with POROS® 50 HS resin. The virus inactivated eluate from the previous Protein A step is adjusted to pH 5.0 and possibly filtered before loading onto the column. After washing with equilibration solvent, the product is eluted in a salt gradient. HCP, media contaminants and aggregates are reduced.

Virusfiltration is performed as a dead end filtration on a Planova 20 N virusfilter to remove potential virus from CHO-cells in which the mAb is expressed.

The Q membrane step is a flow-through step in which the diluted product passes through during load and primarily DNA, endotoxins, HCP and other impurities are reduced.

The HIC step is introduced to reduce the dimer and pre-monomer content. The product from the Q membrane step is added ammonium sulphate and loaded on a HIC column. The elution is effected by a gradient from high to low ammonium sulphate concentration.

The last step is UF/DF in which the product is concentrated by TFF and diafiltrated with API formulation buffer including sucrose. The Tween 80 is added and the API product is filtered through 0.22 μm filter.

### SUMMARY

The results from various experiments are summarized in table 5 here below, showing that in all tests the HIC step enables reduction of the concentration of antibody aggregates providing an antibody composition suitable for further use in a pharmaceutical product or formulation.

TABLE 5
Comparison of the results

| | Protein A | Preparation | |
|---|---|---|---|
| | purified and | including CIEX | Preparation |
| | virus | purification | including |
| | inactivated | (intervals | CIEX and |
| | antibody | covering three | HIC |
| | preparation | experiment) | purification |
| HMWP total (%) | 4.9 | 1.7-2.8 | 1.08 |
| Pre-monomer (%) | 0.8 | 0.7-1.0 | 0.69 |
| Dimer (%) | 2.5 | 1.0-1.8 | 0.36 |
| Polymer (%) | 1.7 | 0 | 0 |

1. A method for removing antibody pre-monomers from an aqueous recombinant antibody solution, wherein said method comprises a step of separating the pre-monomers and optionally antibody dimers from monomeric antibodies using hydrophobic interaction chromatography (HIC).

2. A method according to claim 1, comprising the steps of
i) obtaining an antibody preparation comprising a pre-monomer component,
ii) loading said antibody preparation to a hydrophobic interaction chromatography (HIC) column,
iii) allowing binding of the antibody and the pre-monomer to the column material,
iv) sequentially eluting the antibody components of the preparation, and
v) selecting eluate fractions that do not comprise (substantial amount of) the pre-monomer and thereby obtaining a composition of monomeric antibodies.

3. The method according to claim 2, wherein the monomeric antibodies are obtained in the fractions eluting prior to the fractions including a high content of pre-monomer.

4. The method according to claim 2, wherein the monomeric antibodies are obtained in the fractions eluting prior to the fractions including the majority of pre-monomers and antibody dimers.

5. The method according to claim 2, wherein the pre-monomer is removed by deselecting eluate fractions when the accumulation of pre-monomers and other aggregates reaches around 1%.

6. The method according to claim 1, wherein said antibody is an IL-21 antibody.

7. The method according to claim 1, wherein the HIC column material has a binding capacity of 50 g antibody/L or more and/or wherein the HIC column material comprises 4-8% cross-linked agarose comprising a covalently coupled phenyl group and/or wherein the HIC column material comprises 20-45 μMol phenyl groups/ml.

8. The method according to claim 1, wherein said method comprises the step of loading said antibody on the HIC column in the presence of an ammonium sulphate concentration of 0.8-1.5 mol/kg, and subsequently eluting said antibody from the HIC column with a decreasing ammonium sulphate gradient.

9. The method according to claim 8, wherein the monomeric antibody is obtained in the eluate fractions obtained when the ammonium sulphate concentration is decreasing from 0.8 mol/kg to 0.4 mol/kg.

10. The method according to claim 1, wherein said method results in reduction of virus particles.

11. A method according to claim 1, wherein said method comprises the following steps:
a. expressing an antibody in a host cell, wherein said host cell comprises a vector encoding said antibody,
b. collecting cell media comprising said antibody from step (a),
c. binding antibodies from the cell media obtained in step (b) on a protein A affinity column, and eluting said antibodies 10 mmol formic acid/kg at pH 3.5,
d. loading the eluate obtained in step (c) on a cation exchange chromatography column (CIEX), and eluting said antibodies with a NaCl gradient,
e. subjecting the eluate obtained in step (d) to virus filtration,
f. pumping the filtered eluate obtained in step (e) on a flow through membrane,
g. loading the filtered eluate obtained in step (f) on a hydrophobic interaction chromatography column in the presence of 0.8-1.5 mol/kg ammonium sulphate concentration, and
h. eluting said antibodies with a decreasing ammonium sulphate gradient,
i. concentrating said antibodies in the eluate obtained in step (g) by ultrafiltration and diafiltration, and
j. adding surfactant to the concentrated eluate obtained in step (h).

12. A method for preparing an antibody composition comprising at most 1% antibody aggregates (HMWP total), comprising removing antibody pre-monomers from an aqueous recombinant antibody solution by using hydrophobic interaction chromatography (HIC).

13. The method according to claim 12, wherein pre-monomers and optionally antibody dimers are separated from monomeric antibodies using hydrophobic interaction chromatography (HIC).

14. (canceled)

15. A pharmaceutical product obtained by a process according to claim 1.

16. The method according to claim 6, wherein the IL-21 antibody comprises the three CDR sequences as set forth in SEQ ID NO: 2 and the three CDR sequences as set forth in SEQ ID NO: 3.

17. The method according to claim 2, wherein the HIC column material has a binding capacity of 50 g antibody/L or more and/or wherein the HIC column material comprises 4-8% cross-linked agarose comprising a covalently coupled phenyl group and/or wherein the HIC column material comprises 20-45 μMol phenyl groups/ml.

18. The method according to claim 2, wherein said method comprises the step of loading said antibody on the HIC column in the presence of an ammonium sulphate concentration of 0.8-1.5 mol/kg, and subsequently eluting said antibody from the HIC column with a decreasing ammonium sulphate gradient.

19. The method according to claim 18, wherein the monomeric antibody is obtained in the eluate fractions obtained when the ammonium sulphate concentration is decreasing from 0.8 mol/kg to 0.4 mol/kg.

20. The method according to claim 2, wherein said antibody is an IL-21 antibody.

21. The method according to claim 20, wherein the IL-21 antibody comprises the three CDR sequences as set forth in SEQ ID NO: 2 and the three CDR sequences as set forth in SEQ ID NO: 3.
